(19) United States
(12) Patent Application Publication
(10) Pub. No.: US 2017/0105914 A1
(43) Pub. Date: Apr. 20, 2017

(54) ALUMINIUM LACTATE AS A SKIN PROTECTION COMPONENT

(71) Applicant: DEB IP LTD, DENBY, DERBYSHIRE (GB)

(72) Inventors: THOMAS MANGEN, Dusseldorf (DE); BIRGIT TOBER, Kerken (DE); GEORG SCHICK, Krefeld (DE); MARCEL VEEGER, GOCH (DE); NICOLE KAMPS, Krefeld (DE)

(73) Assignee: DEB IP LTD, DENBY, DERBYSHIRE (GB)

(21) Appl. No.: 15/311,282

(22) PCT Filed: May 21, 2015
(86) PCT No.: PCT/GB2015/051508

§ 371 Date: Nov. 15, 2016

(30) Foreign Application Priority Data
May 26, 2014 (DE) 102014209988.9

(51) Int. Cl. A61K 8/26; A61Q 17/00; A61K 8/365

(52) U.S. Cl. CPC A61K 8/26; A61K 8/365; A61Q 17/00; A61K 2800/74

(57) ABSTRACT

The invention lies within the area of cosmetic and/or dermatological compositions and concerns skin protection agents comprising aluminium lactate and the use of aluminium lactate in skin protection agents. The invention further concerns methods for protecting the skin with skin protection agents comprising aluminium lactate and its uses.

The invention lies within the area of cosmetic and/or dermatological compositions and concerns skin protection agents comprising aluminium lactate and the use of aluminium lactate in skin protection agents. The invention further concerns methods for protecting the skin with skin protection agents comprising aluminium lactate and its uses.

With a surface area of approx. 1.5 to 2.0 m² the human skin is the largest human organ and has to carry out functions that are vitally important for the body. The skin contains blood and lymph vessels for this purpose, through the walls of which lymph fluid, gases, nutrients and waste material are exchanged in order to, for example, guarantee nourishment and metabolism. Further functions of the skin are the regulation of body temperature, the protection of the body against dehydration and against external mechanical, chemical and bacterial influence. The secretions of sebaceous skin glands for example keep the skin supple and help with the regulation of the skin's water content. In addition the skin transmits taste, heat, cold and pain sensations to the organism via free nerve ends, and therefore has the function of a sensory organ.

As a body envelope the human skin provides the connection with, but at the same time also the limitation of the human body against its surroundings. Contact with everything the human organism requires in order to live—but also things that can endanger it—takes place via the same.

It is therefore especially important to look after the human skin and to protect it against environmental influences. The history of skin care products can be traced back far into human developmental history. As requirements placed on skin care products can vary substantially, the range of different skin care products has got ever greater over the last few years in order to be able to adapt to the various needs of the consumer and different loads placed on the skin with regard to the relevant environment. Skin care products for use at work in particular are increasingly becoming subject to greater requirements that exceed the range of common household skin care products by far. According to W. Dicke (see W. Dicke; I. Funk-Stendel; B. Marschner, F. Zuther: All about skin protection, skin cleaning, skin care. 5^{th} updated and extended volume (115-118); October 2005) modern skin care products are preparations that contribute towards the upkeep and replenishment of the physiological hydrolipid emulsion in the upper skin layers. Skin care products often contain special ingredients that support the regeneration process of the skin. Products for the care and/or maintenance of the skin's health as well as skin protection products must for example be adapted to suit the requirements of the workplace and the degree of strain placed on the skin.

As the upper skin layers can swell substantially following the repeated washing of hands or when working in a humid environment, the stressed skin will lose moisture, bodily fats and nutrients through swollen pores. The skin will therefore become chapped. The frequent or regular wearing of gloves, for example latex gloves that are for example worn when working in professions within the medical sector in order to comply with hygiene regulations, or within the chemical industry for protection against toxins in particular, will place an above average strain on the skin.

According to prior art such problems can for example be solved with the aid of natural tannins such as willow bark extracts or witch-hazel extracts. Thanks to the astringent effect of these tannins the skin will swell less when working in a humid environment, and will therefore become more resilient. According to current knowledge astringent agents will change specific body proteins. This can for example enable the formation of a protective layer over wounds and mucous membranes, which supports healing. One huge disadvantage of these natural tannins, such as willow bark extracts or witch-hazel extracts, described in prior art is their relatively low effectiveness together with a simultaneous strong product discolouration. Synthetic tannins in cosmetic formulations also lead to massive changes in product colours, in particular during storage periods of several weeks to months.

The skin protection at work sector in particular, and especially work within a humid environment according to TRGS 401 “Skin Contact Risks”, necessitates high effectiveness of the astringent agents used as well as sustained protection of the skin. Areas of skin permanently or regularly covered with air-impermeable and/or non-breathable materials over periods from several minutes to several hours will lose resilience, skin integrity is compromised, and it will become chapped. People whose job regularly requires the wearing of air-impermeable and/or non-breathable clothing, such as for example gloves, arm sleeves, foot coverings, upper body clothing, leg coverings, trousers or whole body suits, for example made of neoprene, latex, rubber or vinyl, are affected particularly severely by negative effects on the skin. Severe job-related stresses placed on the skin do for example occur within the sector of the medical professions and other professions that require the wearing of protective latex gloves, which pose a great challenge with regard to skin protection.

There is therefore a continued need for skin care products that will satisfy the high demands of the consumer with regard to care and protection on the one hand, and that will not negatively affect product characteristics, in particular with regard to the product colour, on the other.

Aluminium lactate is known as an ingredient of deodorant products and of mouth and dental care products within the cosmetics sector.

It was therefore the purpose of the present invention to provide skin care products that protect the skin against swelling and dehydration, and that will not negatively affect the characteristics of the product, in particular with regard to the product colour, on the other hand.

It has surprisingly been found that aluminium lactate, in particular in combination with sodium lactate, will prevent or at least reduce a swelling of the skin without however discolouring the formulation in cream formulations for use on the skin and for skin care purposes. The skin will also become more resilient towards mechanical strain such as, for example, when working with insulation material such as fibreglass, wood wool, mineral wool. Cream formulations according to the invention also act in a preventative way against skin dehydration and a chapped feeling of the skin, for example caused by frequent work within a humid environment. Skin protection products according to the invention also produce a pleasant, dry feeling whilst working with air-impermeable and/or non-breathable materials such as for example latex gloves, on the skin. The use of aluminium lactate in cosmetic skin protection agents and for cosmetic use can therefore rectify the disadvantages of prior art described above.

The objects of the present invention are therefore skin protection agents comprising aluminium lactate.

The objects of the invention will be described below by way of examples without intending to limit the invention to these examples of embodiments. Wherever ranges, general formulas or compound classes are listed hereafter, these shall include not only the corresponding ranges or groups of compounds explicitly mentioned, but also all part ranges and part groups of compounds that could be obtained through removing individual values (ranges) or compounds. Wherever documents are cited as part of the present description their content, in particular with regard to the facts in the connection with which the document was cited, these shall form part of the disclosure content of the present invention. Percentage information will, unless stated differently, represent weight percent values. pH values are, unless stated differently, determined with the aid of commercially available pH meters based on potentiometry. Wherever median values are stated hereafter these are, unless stated differently, median weights. Wherever parameter determined through measurement are listed hereafter these measurements were, unless stated differently, recorded at a temperature of 25° C. and a pressure of 101.325 Pa.

One preferred object of the present invention is skin protection agents comprising a combination of aluminium lactate and sodium lactate. It has been found that the use of a combination consisting of aluminium lactate and sodium lactate in particular will result in skin protection agents comprising particularly good characteristics. With longer and repeated wearing of gloves made from polymer materials such as latex the prior use of a skin protection agents comprising a combination of aluminium lactate and sodium lactate clearly reduces swelling of the skin and thus sustainably supports the resilience of the skin. At the same time skin protection agents comprising a combination of aluminium lactate and sodium lactate are storage-stable and colour-stable for long periods of time, and therefore do not cause discolouration. The characteristics described above can in particular be realised with skin protection agents comprising a combination of aluminium lactate and sodium lactate at a weight ratio of aluminium lactate to sodium lactate of 1:1 to 500:1, preferably of 300:1 to 1.2:1, particularly preferably of 1.5:1 to 100:1, further preferably of 2:1 to 50:1, and in particular preferably of 6:1 to 30:1. Such skin protection agents in particular lead to measurably lower moisture in the upper skin layers and a pleasant dry feeling on the skin during contact with the skin prior to, or whilst wearing air-impermeable and/or non-breathable materials on these skin sections. In a preferred embodiment skin protection agents according to the invention therefore comprise a combination of aluminium lactate and sodium lactate at a weight ratio of aluminium lactate to sodium lactate of 1:1 to 500:1, preferably of 300:1 to 1.2:1, particularly preferably of 1.5:1 to 100:1, further preferably of 2:1 to 50:1, and in particular preferably of 6:1 to 30:1.

Particularly preferably skin protection agents according to the present invention comprise aluminium lactate in quantities of 1 to 50 wt. %, particularly preferably 3 to 40 wt. %, further preferably 5 to 15 wt. %, and in particular preferably 7 to 12 wt. %, in relation to the total weight of the skin protection agents. If a combination of aluminium lactate and sodium lactate is used in the skin protection agents according to the invention, then the total quantity of aluminium lactate and sodium lactate is preferably 1 to 50 wt. %, particularly preferably 3 to 40 wt. %, further preferably 5 to 15 wt. %, and in particular preferably 7 to 12 wt. %, in relation to the total weight of the skin protection agents. Aluminium lactate and sodium lactate are preferably used in the quantities stated and in the weight ratios described above, as these skin protection agents achieved particularly excellent results during use.

Apart from a combination of aluminium lactate and sodium lactate, particularly preferable skin protection agents comprise further components, which are listed hereafter:

In a preferred embodiment the skin protection agents further comprise at least one emollient. An emollient in the sense of the present invention can in principle be understood as all active substances known to the person skilled in the art, that will positively influence the sensors of a composition, and that will for example make the epidermis of the skin suppler and softer. Preferred emollients can be selected from the group of PEG-30 glyceryl cocoate, PEG-6 caprylic/capricyc glyceride, sorbitan sesquicaprylicate, sucrose cocoate, isoamyl cocoate, polyglyceryl-3 caprate, PPG-3 myristil ether, PPG-11 stearyl ether, cetearyl isononanoate, cetyl ethylhexanoate, caprylicic/capricyc triglyceride, decal cocoates, diethylhexyl carbonates, decyl oleates, PPG-15 stearyl ether, octyldodecanol, isocetyl palmitate, isohexadecane, cetearyl ethylhexanoate, isopropyl myristat, oleyl erucate, ethylhexyl palmitate, ethylhexyl stearate, isopropyl palmitate, PPG-14 butyl ether, triisostearine, C12-15 alkylbenzoate, cetyl ricenoleate, glyceryl ricenoleate, glyceryl stearate, isocetyl palmitate, isocetyl stearate, tocopheryl linoleate, methylheptyl isostearate and mixtures thereof.

In a preferred embodiment the skin protection agents further comprise at least one linear, saturated unsubstituted fatty alcohol with 16 to 18 carbon atoms, preferably selected from stearyl alcohol and cetyl alcohol.

The skin protection agents can further preferably comprise additional components, preferably selected from polyglyceryl-3 methyl glucose distearate, polyglyceryl-6 stearate, polyglyceryl-6 isopropyl palmitate and/or glycerine.

Particularly preferable is a combination of aluminium lactate and sodium lactate and at least one further component selected from polyglyceryl-6 stearate, polyglyceryl-6 isopropyl palmitate, isocetyl palmitate polyglyceryl-3 methyl glucose distearate, stearyl alcohol, cetyl alcohol and glycerine, as such formulations according to the invention have particularly good skin protection characteristics.

Preferred compositions according to the invention comprise at least 60 wt. %, preferably at least 70 wt. %, particularly preferably at least 80 wt. % water.

Preferred compositions comprise as an active substance combination, in relation to the total weight of the composition,
a) 1 to 50 wt. % of a combination of aluminium lactate and sodium lactate at a weight ratio of aluminium lactate to sodium lactate of 1:1 to 500:1;
b) 0 to 1 wt. %, preferably 0.05 to 0.3 wt. % of at least one linear, saturated unsubstituted fatty alcohol with 12 to 20 carbon atoms, selected from stearyl alcohol and cetyl alcohol;
c) 3 to 30 wt. %, preferably 5 to 15 wt. %, of at least one emollient, preferably selected from PEG-30 glyceryl cocoate, PEG-6 caprylic/capricyc glyceride, sorbitan sesquicaprylicate, sucrose cocoate, isoamyl cocoate polyglyceryl-3 caprate, PPG-3 myristil ether, PPG-11 stearyl ether, cetearyl isononanoate, cetyl ethylhexanoate, caprylicic/capricyc triglyceride, decal cocoates, diethylhexyl carbonates, decyl oleates, PPG-15 stearyl ether, octyldodecanol, isocetyl palmitate, isohexadecane, cetearyl ethylhexanoate, isopropyl myristate, oleyl erucate, ethylhexyl palmitate, ethylhexyl stearate, isopropyl palmitate, PPG-14 butyl ether, triisostearine, C12-15 alkylbenzoate, cetyl ricenoleate, glyceryl ricenoleate, glyceryl stearate, isocetyl palmitate, isocetyl stearate, tocopheryl linoleate, methylheptyl isostearate and mixtures thereof;
d) 0 to 5 wt. %, preferably 0.05 to 3 wt. % glycerine;
f) water;
g) optional further components,
whereby the sum of all weight components is 100 wt. %.

The compositions according to the invention are preferably cosmetic and/or dermatological formulations, which can generally be present as aqueous alcohol solutions, creams, emulsions, lotions, gels, aerosol sprays or foams, non-aerosol sprays or foams. They are preferably creams and/or lotions. Particularly preferably they are hydrophilic creams and/or lotions, which can be present as oil in water emulsions (O/W emulsions).

The compositions according to the invention can optionally comprise further components as are normally used in cosmetic and/or dermatological formulations. Preferred further components can be selected from the group of scents and perfumes, preservatives and/or pH adjusters, such as for example sodium hydroxide solution.

The compositions according to the invention can also comprise further commonly used components for adjustment purposes, which serve for the treatment, care, cleaning and the protection of skin, such as for example active skin cosmetics substances (active ingredients). The term active skin cosmetics substances in the sense of the present invention include, for example, ceramides, pseudoceramides, protein hydrolysates of a vegetable or animal origin based on keratin, collagen, elastin, wheat, rice, soya, milk, silk, maize, amino acids and amino acid derivatives, inflammation inhibiting active substances, antimicrobial active substances, commonly used antioxidants, vitamins, dexpanthenol, lactic acid, pyrrolidoncarboxylic acid, bisabolol and vegetable, yeast and/or algae extracts.

Combination with commonly used organic or inorganic UV filter substances is also possible and can for example be considered as particularly advantageous in sun protection preparations, as a drying out of the skin during use can be effectively avoided and the natural protective function of the skin maintained in this way. Further cosmetic additives and admixtures, commonly used in such preparations, can also be included. Such additives are, for example, solubilisers such as ethanol, isopropanol, ethylene glycol, propylene glycol and diethylene glycol. The further components include cosmetic oils of a vegetable and synthetic origin, silicon oils, fats, refattening agents, emulsifiers, anionic, zwitterionic, ampholytic and non-ionic surfactants and/or colorants.

Lastly, the formulations according to the invention can also comprise chelating agents such as EDTA, NTA, β-alanin diacetic acid and phosphonic acid, colorants for colouring the cosmetic preparation, opacifiers such as latex, styrol/PVP and styrol acrylamide copolymers, ceramide, β-glucanes, oligopepides, hyaluronic acid, pearlescent agents such as ethylene glycol mono-and distearate and PEG-3-distearate, pigments, light protection agents, thickeners or propellants.

In a preferred embodiment compositions according to the invention are free from compounds selected from silicone compounds, dipropylene glycol, parabens and/or alkyl parabens, formaldehyde splitters such as diazolidinyl urea, imidazolidinyl urea and/or DMDM hydantoine, preservatives based on halogen-organic compounds such as for example triclosane and mixtures thereof. Particularly preferable compositions according to the invention are free from parabens and/or alkyl parabens, in particular preferably free from parabens and alkyl parabens. According to the invention the terms parabens and alkyl parabens include in particular also 4-hydroxy benzoic acid and 4-hydroxy benzoic acid ester, such as for example methyl-4-hydroxy benzoate, ethyl-4-hydroxy benzoate, propyl-4-hydroxy benzoate and/or butyl-4-hydroxy benzoate. Also particularly preferred according to the invention are compositions free from silicone compounds. This can in particular be desirable if the compositions are used in a working environment with sensitive surfaces, such as for example surfaces to be painted, as silicone compounds may leave residues on work pieces or products, which can then have a negative effect on the product during further processing. In an especially preferred embodiment compositions according to the invention can be free from silicone compounds and free from parabens and free from alkyl parabens.

The compositions according to the invention can be produced in any way. A further object of the present invention is the production of skin protection agents comprising aluminium lactate. Preferred is the production of skin protection agents comprising a combination of aluminium lactate and sodium lactate, preferably at a weight ratio of aluminium lactate to sodium lactate of 1:1 to 10:1, preferably of 2:1 to 8:1, particularly preferably of 2.2:1 to 5:1, and in particular preferably of 2.5:1 to 3.5:1.

A further object of the present invention is a method for protecting skin, whereby a skin protection agent according to the invention as described above, in particular also comprising all of the preferred embodiments of the skin protection agent according to the invention described above, is brought into contact with parts of the human skin, with these parts of the human skin being subsequently or simultaneously covered. The parts of the human skin are preferably covered with air-impermeable and/or non-breathable materials, whereby the materials preferably consists of air-impermeable and/or non-breathable clothing, preferably made of neoprene, latex, rubber or vinyl, in particular gloves, arm sleeves, foot coverings, upper body clothing, leg coverings, trousers or whole body suits. According to the invention the terms “air-impermeable and/or non-breathable materials” include every material that will result in moisture generated through natural transpiration of the skin between the material and the skin not being conducted, or being conducted badly. This effect is for example known from wearing latex gloves. The natural emission of moisture from the skin leads to a damp feeling being created on the hand under the glove and the skin surface becoming moist, or even wet. The skin protection agents according to the invention have proven themselves as particularly good for such methods in particular, and have resulted in measurably less moisture in the upper skin layers and a pleasant dry feeling of the skin in the parts of the skin in question.

A further object of the present invention is the use of aluminium lactate in skin protection agents. A further object of the present invention is the use of aluminium lactate in skin protection agents for improving the firmness and/or the mechanical characteristics of the skin. The regular use of skin protection agents according to the invention results in skin that is more resilient towards mechanical strain and that is firmer, such as for example when working with insulation material such as fibreglass, wood wool, mineral wool. Cracked skin and injuries to the upper skin layers can therefore be prevented when working with sharp, angular or pointed materials. A further object of the present invention is the use of aluminium lactate in cream formulations for stabilising the skin, in particular the upper epidermal layers. The described uses preferably include a combination of aluminium lactate and sodium lactate, particularly preferably at a weight ratio of aluminium lactate to sodium lactate of between 1:1 to 500:1, preferably of 300:1 to 1.2:1, particularly preferably of 1.5:1 to 100:1, further preferably of 2:1 to 50:1, and in particular preferably of 6:1 to 30:1, whilst it is particularly preferable if a combination of aluminium lactate and sodium lactate and at least one further component selected from polyglyceryl-6 stearate, polyglyceryl-6 isopropyl palmitate, isocetyl palmitate polyglyceryl-3 methyl glucose distearate, stearyl alcohol, cetyl alcohol and glycerine is used.

A further object of the present invention is the use of skin protection agents according to the invention when working in wet conditions, preferably prior to and/or whilst wearing air-impermeable and/or non-breathable materials on the skin.

A further object of the present invention is the use of skin protection agents according to the invention for improving grip resistance. The improvement of grip resistance is obvious in particular when using the skin protection agents according to the invention prior to or whilst working or playing sports where the grip resistance of the palms is important, such as for example when working with a hammer or playing tennis, golf or other types of sport where a racket is used. Improved grip resistance according to the invention exists when the subjective perception of the corresponding test person confirms this.

A further object of the present invention is the use of aluminium lactate for topical use on the skin. A further object of the present invention is the use of aluminium lactate in cream formulations for skin care purposes.

Skin protection agents comprising aluminium lactate and/or compositions according to the invention are preferably applied to the human skin as a cream, lotion or gel. A further object of the present invention is the use of skin protection agents according to the invention comprising aluminium lactate and/or compositions according to the invention for topical application to the skin. Any part of the skin, such as for example the face, hands, lower arms and the entire human body can preferably be treated with skin protection agents comprising aluminium lactate and/or the compositions according to the invention. The hands, lower arms and the face, in particular the hands and lower arms, are preferably treated with skin protection agents comprising aluminium lactate and/or the compositions according to the invention. These include all possible forms of application onto the skin according to the invention. Examples of possible applications are the application by hand and with application aids as well as smoothing on, rubbing in, spraying on or massaging in.

A further object of the present invention is the use of skin protection agents according to the invention comprising aluminium lactate and/or compositions according to the invention as a skin cream, in particular as a cream for hands and lower arms.

A further object of the present invention is the cosmetic use of skin protection agents according to the invention comprising aluminium lactate and/or compositions according to the invention for skin care purposes. A further object of the present invention is the cosmetic use of skin protection agents according to the invention comprising aluminium lactate and/or compositions according to the invention for skin protection purposes. A further object of the present invention is the cosmetic use of skin protection agents according to the invention comprising aluminium lactate and/or compositions according to the invention for protecting the skin against dehydration, in particular whilst wearing gloves/protective gloves made of plastic, such as for example latex.

A further object of the present invention is the cosmetic use of skin protection agents according to the invention comprising aluminium lactate and/or compositions according to the invention for reducing a rough, dry and/or tight feeling of the skin. A reduction of a rough, dry and/or tight feeling of the skin exists according to the invention when the subjective perception of the corresponding test person confirms this.

The invention will now be explained with reference to examples. These explanations represent mere examples and are not intended to limit the present invention in any way.

### EXAMPLES

### Example 1

### Compositions of Skin Protection Agents According to the Invention

| | A | B | C | D | E | | | | |
|---|---|---|---|---|---|---|---|---|---|
| Raw materials | O/W | O/W | O/W | O/W | O/W | F | G | H | I |
| (INCI) | emulsion | emulsion | emulsion | emulsion | emulsion | Spray | Spray | Spray | Spray |
| Water | 82.24 | 79.96 | 76.36 | 65.36 | 52.86 | 63.20 | 69.20 | 55.20 | 39.20 |
| Glycerine | 3.000 | 3.000 | 3.000 | 3.000 | 3.000 | | | | |
| Sodium | 0.600 | 0.600 | 0.600 | 0.600 | 0.600 | 0.600 | 0.600 | 0.600 | 0.600 |
| benzoate | | | | | | | | | |
| Lactic acid | 0.200 | 0.200 | 0.200 | 0.200 | 0.200 | | | | |
| Aluminium | 0.900 | 3.000 | 6.000 | 15.000 | 25.000 | 30.000 | 30.000 | 30.000 | 30.000 |
| lactate | | | | | | | | | |
| Sodium lactate | 0.420 | 0.600 | 1.200 | 3.000 | 5.000 | 6.000 | 1.000 | 15.000 | 30.000 |
| Xanthan gum | 0.400 | 0.400 | 0.400 | 0.400 | 0.400 | | | | |
| Dehydroxanthan | 0.250 | 0.250 | 0.250 | 0.250 | 0.250 | | | | |
| gum | | | | | | | | | |
| Polyglyceryl-6 | 1.370 | 1.370 | 1.370 | 1.370 | 1.370 | | | | |
| stearate | | | | | | | | | |
| Polyglyceryl-6 | 0.120 | 0.120 | 0.120 | 0.120 | 0.120 | | | | |
| behenate | | | | | | | | | |
| Polyglyceryl-3 | 3.500 | 3.500 | 3.500 | 3.500 | 3.500 | | | | |
| methyl glucose | | | | | | | | | |
| distearate | | | | | | | | | |
| Cetylstearyl | 0.800 | 0.800 | 0.800 | 1.000 | 1.500 | | | | |
| alcohol | | | | | | | | | |
| Isopropyl | 2.000 | 2.000 | 2.000 | 2.000 | 2.000 | | | | |
| palmitate | | | | | | | | | |
| Isocetyl | 4.000 | 4.000 | 4.000 | 4.000 | 4.000 | | | | |
| palmitate | | | | | | | | | |
| Perfume | 0.200 | 0.200 | 0.200 | 0.200 | 0.200 | 0.200 | 0.200 | 0.200 | 0.200 |
| | 100.000 | 100.000 | 100.000 | 100.000 | 100.000 | 100.000 | 100.000 | 100.000 | 100.000 |

Unlike aluminium chlorohydrate, which is often used in deodorant formulations, the skin protection agents according to the invention, comprising aluminium lactate, in particular aluminium lactate and sodium lactate, as components of the natural moisturising factor, offer the positive effect that although the upper skin layers are cross-linked and lower capacitive conductivities are therefore measured (corneo meter tests), the skin as a whole will not dry out (sensors).

It has surprisingly been found that even a use of the skin protection agents A to I on intact skin will generate efficient protection. This makes the skin protection agents according to the invention valuable for use as skin protection formulations under air-impermeable and/or non-breathable materials, such as for example gloves.

### Example 2

### Precipitation Trials

The astringent characteristics of the skin protection agents according to the invention were confirmed by means of precipitation trials.

The precipitation reaction of a Bovine Serum Albumin (BSA) solution with aluminium lactate solution and aluminium lactate/sodium lactate solution in different concentrations determines the reduction of absorption intensity within the UV-VIS range caused by this precipitation (“tanning”) in centrifugation supernatants of the precipitation reactions as proof of the tanning effect in a photometric way. Floctan 1 solutions with corresponding concentrations are compared as the standard for known “tannins”.

### Example 3

### Skin Moisture Measurement

Trial Description:

Measurements are taken in an air-conditioned room at a temperature of 21±1.5° C. and a relative humidity of 40±4%. Test persons entered the air-conditioned room 15 minutes prior to the first measurement in order to acclimatise the skin to the ambient temperature and relative humidity. 2 fields each were then marked on the left and right volar lower arms. The moisture content starting values were then determined for these fields with a corneometer CM 825. Product 1 was then rubbed into both fields of the left lower arm, and product 2 into both fields of the right lower arm. The quantity per field equalled the volume of one 9×5 mm spoon spatula. Further skin moisture content measurements followed 15 and 30 minutes after application. The starting values prior to application were then subtracted from the measured values and the median value calculated from the test person measurements.

Number of Test Persons: 4

Product 1—comparison product of composition C from example 1 without aluminium lactate and sodium lactate

Product 2—skin protection agent C from example 1 with 6 wt. % aluminium lactate and 1.2 wt. % sodium lactate

Results:

| | 15 min | 30 min |
|---|---|---|
| Field 1 product 1 | 12.5 | 10.1 |
| Field 2 product 1 | 15.5 | 13.4 |
| Field 3 product 2 | −0.9 | −2.1 |
| Field 4 product 2 | 2.4 | 3.0 |

The comparison product without aluminium lactate and sodium lactate (product 1) clearly increases skin moisture, whilst skin protection agent C with aluminium lactate and sodium lactate (product 2) according to the invention does not.

### Example 4.1

### Sensors

Implementation:

The test persons pull a nitrile glove onto their left hand and rub 0.5 g of product 1 onto their right hand with this. The glove is then removed and the person waits for 30 seconds, so that product 1 has been absorbed into the skin. A nitrile glove is then pulled onto the right hand and 0.5 g of product 2 rubbed into the left hand and the glove removed. Skin feeling is then compared after a further 3 minutes.

Number of test persons: 9

Product 1—comparison product of composition C from example 1 without aluminium lactate and sodium lactate

Product 2—skin protection agent C from example 1 with 6 wt. % aluminium lactate and 1.2 wt. % sodium lactate

Results:

100% of test persons described the skin feel of the skin protection agent C with aluminium lactate and sodium lactate (product 2) according to the invention as “drier” and perceived the same as pleasant compared to the comparison composition C without aluminium lactate and sodium lactate (product 1).

Almost all of the test persons described the skin feel of comparison composition C without aluminium lactate and sodium lactate (product 1) as “moister”.

The vast majority described the skin feel of the skin protection agent C with aluminium lactate and sodium lactate (product 2) according to the invention as “more grippy” and the comparison product without aluminium lactate and sodium lactate (product 1) as “greasier”.

### Example 4.2

### Sensors

Examination of skin feel after 3 h of wearing gloves.

Implementation:

The test persons pull a nitrile glove onto their left hand and rub 0.5 g of product 1 onto their right hand with this. The glove is then removed and the person waits for 30 seconds, so that product 1 has been absorbed into the skin. A nitrile glove is then pulled onto the right hand and 0.5 g of product 2 rubbed into the left hand. A further nitrile glove is then also pulled over the left hand, onto which cream has been applied. The gloves are worn on both hands for 3 hours and then removed. A comparison between the left and right hands is then carried out.

Number of test persons: 3

Products:

Product 1—comparison product of composition C from example 1 without aluminium lactate and sodium lactate

Product 2—skin protection agent C from example 1 with 6 wt. % aluminium lactate and 1.2 wt. % sodium lactate

Results:

100% of test persons were able to tell a difference between the two products and perceived the skin feel of the hand to which the skin protection agent C (product 2) according to the invention was first applied as more pleasant.

In addition to a positively perceivable skin feel it was possible to show that the skin was clearly more resilient against mechanical abrasion of the upper skin layers (displacement in relation to a hard surface—for example “rubbing”) following prior application of the skin protection agent C with aluminium lactate and sodium lactate (product 2) than it was following prior application of product 1. The “eraser effect” in particular is absent following an application of product 2.

1. Skin protection agent, characterised in that it contains aluminium lactate.

2. Skin protection agent according to claim 1, characterised in that the skin protection agent contains a combination of aluminium lactate and sodium lactate.

3. Skin protection agent according to claim 2, characterised in that the skin protection agent contains a combination of aluminium lactate and sodium lactate at a weight ratio of aluminium lactate to sodium lactate of 1:1 to 500:1, preferably of 300:1 to 1.2:1, particularly preferably of 1.5:1 to 100:1, further preferably of 2:1 to 50:1, and in particular preferably of 6:1 to 30:1.

4. Skin protection agent according to claim 2, comprising a total quantity of aluminium lactate and sodium lactate is preferably 1 to 50 wt. %, particularly preferably 3 to 40 wt. %, further preferably 5 to 15 wt. %, and in particular preferably 7 to 12 wt. %, in relation to the total weight of the skin protection agent.

5. Skin protection agent according to claim 1, comprising a skin protection agent further contains at least one additional component selected from polyglyceryl-6 stearate, polyglyceryl-6 isopropyl palmitate, isocetyl palmitate polyglyceryl-3 methyl glucose distearate, stearyl alcohol, cetyl alcohol and glycerine.

6. Method for protecting the skin, comprising a skin protection agent according to claim 1 is brought into contact with parts of the human skin, and in that these parts of the human skin are covered subsequently or simultaneously.

7. Method according to claim 6, characterised in that the parts of the human skin are covered with an air-impermeable and/or non-breathable materials, whereby said material is preferably air-impermeable and/or non-breathable clothing, preferably made of neoprene, latex, rubber or vinyl, in particular gloves, arm sleeves, foot coverings, upper body clothing, leg coverings, trousers or whole body suits.

8. Use of aluminium lactate in skin protection agents.

9. Use of aluminium lactate in skin protection agents for improving the firmness and/or the mechanical characteristics of the skin.

10. Use of aluminium lactate in cream formulations for stabilising the skin, in particular the upper epidermal layers.

11. Use according to claim 8, comprising a combination of aluminium lactate and sodium lactate is used.

12. Use according to claim 11, characterised in that a combination of aluminium lactate and sodium lactate is used at a weight ratio of aluminium lactate to sodium lactate of 1:1 to 500:1, preferably of 300:1 to 1.2:1, particularly preferably of 1.5:1 to 100:1, further preferably of 2:1 to 50:1, and in particular preferably of 6:1 to 30:1.

13. Use according to claim 11, comprising a combination of aluminium lactate and sodium lactate and at least one further component selected from polyglyceryl-6 stearate, polyglyceryl-6 isopropyl palmitate, isocetyl palmitate polyglyceryl-3 methyl glucose distearate, stearyl alcohol, cetyl alcohol and glycerine is used.

14. Use of skin protection agents according to claim 1, whilst working in wet conditions, preferably prior to and/or whilst wearing an air-impermeable and/or non-breathable material on the skin.

15. Use of skin protection agents according to claim 1 for improving the grip resistance.
